(19) United States
(12) Patent Application Publication
(10) Pub. No.: US 2023/0177681 A1
(43) Pub. Date: Jun. 8, 2023

(54) METHOD FOR DETERMINING AN ABLATION REGION BASED ON DEEP LEARNING

(71) Applicant: Quantum Surgical, Montpellier (FR)

(72) Inventors: Estanislao OUBEL, Montpellier (FR); Lucien BLONDEL, Montpellier (FR); Bertin NAHUM, Castelnau-le-Lez (FR); Fernand BADANO, Lyon (FR); Michael GIRARDOT, Castelnau-le-lez (FR)

(21) Appl. No.: 17/925,755

(22) PCT Filed: May 20, 2021
(86) PCT No.: PCT/FR2021/050907

§ 371 Date: Nov. 16, 2022

(30) Foreign Application Priority Data
May 20, 2020 (FR) FR2005340

(51) Int. Cl. G06T 7/00; A61B 34/10

(52) U.S. Cl. CPC G06T 7/0012; A61B 34/10; G06T 2207/20021

(57) ABSTRACT

The invention relates to a method for evaluating in post-treatment an ablation of a portion of an anatomy of interest of an individual, the anatomy of interest comprising at least one lesion. The evaluation method comprises in particular a step of automatically determining a contour of the ablation region by means of an automatic learning method, such as a neural network, analyzing the post-treatment image of the anatomy of interest of the individual, said automatic learning method being preloaded during a so-called training phase using a database comprising a plurality of post-operative medical images of an anatomy of identical interest of a set of patients, each medical image of the database being associated with an ablation region of the anatomy of interest of said patient. The invention also relates to an electronic device comprising a processor and a computer memory storing instructions of such an evaluation method.

### TECHNICAL FIELD OF THE INVENTION

The field of the invention is that of the evaluation of a medical intervention.

More precisely, the invention relates to a method for the post-treatment evaluation of a region of ablation of a lesion and prediction of an associated risk of recurrence.

The invention is applicable in particular to the evaluation of a minimally invasive medical intervention with prediction of a risk of recurrence of a lesion, such as a tumor or a metastasis. Such a minimally invasive medical intervention corresponds, for example, to percutaneous ablation of a lesion, for example a tumor in the liver, a lung, a kidney or any other organ. Percutaneous ablation usually involves image-guiding the insertion of one or more needles through the skin to reach and destroy a lesion.

### PRIOR ART

Techniques for evaluating the effectiveness of an intervention and predicting a risk of recurrence of a lesion are known from the prior art.

Such a technique consists, for example, following the ablation of a lesion, in determining to what extent the ablation region covers the lesion. By comparing the volume of the ablation region with the volume of the lesion, it is possible to determine the ablation margins. In practice, it is generally recommended to have ablation margins of at least five millimeters.

In order to determine these margins, the volume of the lesion is usually determined when planning the intervention and compared with the volume of the ablation region which is segmented by an operator on at least one post-operative image.

The main drawback is that the volume of the ablation region is generally determined with little precision, in a way that is often dependent on the operator who performed the segmentation. In addition, the quality of post-operative images is often poor, which contributes to the introduction of uncertainties into the segmentation. Therefore, a correlation between ablation margins and a risk of recurrence of a lesion is difficult to establish.

In order to improve on the techniques of the prior art, it is known practice to use methods for automatically segmenting the ablation region.

Such a technique is, for example, described in the scientific publication by Zhang et al. entitled “Detection and Monitoring of Thermal Lesions Induced by Microwave Ablation Using Ultrasound Imaging and Convolutional Neural Networks”, published in September 2019. The segmentation method described in this publication makes it possible to calculate the margins of the ablation region by segmenting a pre-operative ultrasound image and a post-operative ultrasound image.

However, the segmentation method described in this publication does not make it possible to predict the risk of recurrence because the precision of the automatic segmentation of the ablation region is low. First, the segmentation method is limited to a sub-sampling matrix of an image of the ablation region, the size of which is fixed, generally equal to 4 mm², thus limiting the use of the method to regions of small size. In addition, it is necessary to know the position of the ablation region in order to fix the position of the sub-sampling matrix, making the method difficult to use in the absence of constant reference points inside the sub-sampling matrix in the pre-operative and post-operative images. Lastly, due to the nature and quality of two-dimensional ultrasound images, the anatomical structure of interest may be difficult to discern, making segmentation of the region imprecise, resulting, in particular, in erroneous observations where the ablation region as segmented does not encompass the lesion to be ablated.

In addition, automatic segmentation methods give consistent results for homogeneous regions, i.e. for a bone, blood vessels, or a lesion, for example, or when the image comprises a known number of regions. In the case of ablation regions, the segmentation results obtained are not very consistent because ablation regions are highly complex regions generally composed of different materials such as gas, necrotic cells, healthy cells, residual contrast agent, calcification, etc. In addition, segmentation is generally performed on medical images that are generally blurry and of low contrast, making automatic segmentation of the image difficult.

None of the current systems makes it possible to simultaneously meet all of the required needs, namely to provide a technique that makes it possible to finely evaluate an ablation treatment by segmenting an ablation region, in particular a heterogeneous ablation region, with better precision, in particular on the basis of a blurry and/or low-contrast medical image.

### SUMMARY OF THE INVENTION

The present invention aims to overcome all or some of the drawbacks of the prior art mentioned above.

To that end, one subject of the invention is a method for the post-treatment evaluation of an ablation of a portion of an anatomical structure of interest of an individual, the anatomical structure of interest comprising at least one lesion.

The ablation is a percutaneous or minimally invasive ablation, which generally involves the insertion of at least one needle through the skin to reach and destroy a lesion. A number of ablation techniques are possible: radiofrequency, microwave, electroporation, laser, cryotherapy, ultrasound, etc.

The anatomical structure of interest may be a liver, lung, kidney, or any other organ liable to feature a lesion.

According to the invention, the post-treatment evaluation method comprises steps of:
acquiring a post-operative medical image of the anatomical structure of interest of the individual;
automatically determining an outline of the ablation region via a machine learning method, of neural network type, analyzing the post-treatment image of the anatomical structure of interest of the individual, said machine learning method being trained beforehand in what is called a training phase using a database comprising a plurality of medical images of an identical anatomical structure of interest of a set of patients, each medical image in the database being associated with an ablation region for the anatomical structure of interest of said patient.

Thus, the ablation region is segmented automatically in the post-operative medical image based on prior learning from a plurality of medical images segmented by at least one operator, preferably by a plurality of operators. It should be noted that this automatic segmentation by the neural network makes it possible to dispense with the presence of an operator experienced in the analysis of medical images. In addition, the segmentation obtained by the neural network is generally more precise, in particular in the case of analyzing three-dimensional images. Lastly, the automatic segmentation using such a method is also of better quality in the case of an image exhibiting low contrast and/or sharpness.

In some particular implementations of the invention, the training phase comprises a prior step of training using medical images of an identical anatomical structure of interest comprising an unablated lesion.

Thus, the neural network better segments the ablation region in the post-operative medical image of the individual. This surprising effect may be explained through the similarity in terms of shape and position between the lesion and the ablation region. In addition, it should be noted that the number of accessible medical images showing an unablated lesion on a given anatomical structure of interest is generally higher than the number of accessible medical images acquired after ablation of the lesion.

In other particular implementations of the invention, the post-treatment evaluation method further comprises a step of registering the post-operative image and a medical image of the anatomical structure of interest of the individual, acquired before the surgical treatment, called the the pre-operative medical image, the registered post-operative medical image and pre-operative medical image forming a pair of medical images of the anatomical structure of interest of the individual.

Thus, an analysis of the position of the ablation region in relation to the lesion may be performed.

What is meant by “pre-operative medical image” is a medical image acquired before the ablation treatment and what is meant by “post-operative medical image” is a medical image acquired after the ablation treatment.

In some particular implementations of the invention, the post-treatment evaluation method further comprises a step of evaluating a risk of recurrence according to a relative characteristic between the ablation region and the lesion, between the ablation region and the anatomical structure of interest or between the lesion and the anatomical structure of interest.

Thus, the post-treatment evaluation method according to the invention offers medical staff a better view of the ablation treatment applied to the individual, by allowing them to assess the need for additional treatment if the risk of recurrence is demonstrated.

The risk of recurrence generally takes the form of a binary value, which may, for example, be equal to 0 or 1. A positive value will be understood as the risk of recurrence being demonstrated and a negative value as the risk of recurrence being low.

However, the risk of recurrence may also take the form of a probability between 0 and 1. The risk of recurrence will then be understood as having been demonstrated when the value of the risk is higher than a threshold value, for example equal to 0.5.

In some particular implementations of the invention, when the risk of recurrence is demonstrated, the post-treatment evaluation method further comprises a step of determining the position of the recurrence according to a relative characteristic between the ablation region and the lesion, between the ablation region and the anatomical structure of interest or between the lesion and the anatomical structure of interest.

In some particular implementations of the invention, the risk of recurrence is evaluated by taking account of an ablation margin between the ablation region and the lesion.

For example, the ablation margins are equal to or larger than 5 mm for the value of the risk of recurrence to be negative.

The ablation margin is generally defined as the smallest distance between the ablation region and the lesion.

In some particular implementations of the invention, the risk of recurrence is evaluated by taking account of a distance between a center of mass of the lesion and a center of mass of the ablation region.

The reference value of the center of mass depends on the ablation margins. If the ablation margins are 10 mm and the reference value of the ablation margins is 5 mm, the distance between the centers of mass of the lesion and the centers of mass of the ablation region should be smaller than or equal to 5 mm.

In some particular implementations of the invention, the risk of recurrence is evaluated by taking account of the evenness and the sharpness of the edges of the ablation region in relation to the surrounding healthy tissue.

What is meant by “surrounding healthy tissue” is healthy tissue of the anatomical structure of interest located inside the frame of the cropped medical image.

In some particular implementations of the invention, the risk of recurrence is evaluated by taking account of the ratio of the volume of the lesion to the volume of the ablation region.

In some particular implementations of the invention, the risk of recurrence is evaluated by taking account of a position of the lesion in relation to the center of the anatomical structure of interest.

In some particular implementations of the invention, the post-treatment evaluation method further comprises a step of segmenting the lesion in the pre-operative medical image of the anatomical structure of interest of the individual.

In some particular implementations of the invention, the post-treatment evaluation method further comprises a step of detecting the lesion in the pre-operative medical image of the anatomical structure of interest of the individual.

In some particular implementations of the invention, all or some of the medical images in the database are cropped around the ablation region comprising at least one lesion, the cropping of the images being carried out using a common frame of predetermined size, the set of the centers of the ablation region in the cropped medical images in the database forming a constellation of distinct points inside the common frame.

Thus, by distributing the position of the ablation regions in the common frame, it is possible to decrease prediction errors in the machine learning method. In the case of all of the ablation regions being in the same position in the common frame, the machine learning method would primarily consider post-operative images featuring an ablation region in that particular position, thereby leading to prediction errors in the case of the ablation region being in another position.

In some particular implementations of the invention, for the set of medical images in the database, the portion of the individual’s body included in said image is divided into a plurality of elementary units of a single size, the number of elementary units being divided into two near-equal parts between the portion of the human body delimited by the ablation region and the rest of the portion of the individual’s body included in the image.

It should be noted that the equal distribution between the elementary units corresponding to an ablation region and the elementary units corresponding to a non-ablation region may be analyzed at image level or globally across all of the images.

The elementary units are generally called pixels in the context of two-dimensional images or voxels in the context of three-dimensional images.

What is understood by “near-equal parts” is when the two sets of elementary units consist of the same number of elementary units or when the difference in the number of elementary units in each of the two sets is, for example, smaller than 5% of the number of elementary units in the two sets.

In some particular implementations of the invention, the post-operative medical image database comprises at least one pre-operative medical image comprising at least one unablated lesion.

Thus, the machine learning method is better trained.

In some particular implementations of the invention, the post-treatment evaluation method further comprises a step of determining a supplementary ablation mask when the risk of recurrence is demonstrated.

Thus, a treatment proposal is estimated with a view to eliminating the risk of recurrence via the post-treatment evaluation method. It should be noted that this treatment proposal is not mandatory and might or might not be followed by medical staff.

In some particular implementations of the invention, the post-treatment evaluation method further comprises a step of planning a path for a medical instrument to a target point of an ablation region defined by the supplementary ablation mask.

It should be noted that this planning step is carried out prior to the supplementary treatment.

In some particular implementations of the invention, the post-treatment evaluation method further comprises a step of assisting an operator of the medical instrument in following the planned path.

In some particular implementations of the invention, the planned path and/or a guiding indicator is displayed in real time on a screen of an augmented reality device.

In some particular implementations of the invention, the medical images are three-dimensional images.

It should be noted that a three-dimensional image may correspond to a collection of two-dimensional images taken at generally regular intervals along a predefined axis.

In some particular implementations of the invention, each post-operative image is acquired using the same image acquisition technique.

In other words, the technique used to acquire the post-operative medical image is identical to that used to acquire the post-operative medical images in the training database for the machine learning method.

The invention further relates to an electronic device comprising a processor and a computer memory storing instructions for a method according to any one of the preceding implementations.

Such an electronic device may, for example, be a control device, a navigation system, a robotic device or an augmented reality device. The control device may, in particular, be a computer present in the operating room or a remote server.

### BRIEF DESCRIPTION OF THE FIGURES

Other advantages, aims and particular features of the present invention will become apparent from the following non-limiting description of at least one particular embodiment of the devices and methods which are the subject of the present invention, with reference to the appended drawings, in which:

FIG. 1 is a schematic view of a medical intervention;

FIG. 2 is a block diagram of a method for the post-treatment evaluation of the medical intervention of FIG. 1;

FIG. 3 is an example of a three-dimensional medical image in which an ablation region is highlighted, used in the method illustrated in FIG. 2;

FIG. 4 is an example of four medical images each comprising an ablation mask delimited manually by an operator and an ablation mask predicted by a neural network of the method illustrated in FIG. 2;

FIG. 5 is an example of a medical image in which automatic segmentation of a lesion and of an ablation region is carried out in a step of the method illustrated in FIG. 2;

FIG. 6 illustrates an example of a supplementary ablation as proposed optionally by the method illustrated in FIG. 2.

### DETAILED DESCRIPTION OF THE INVENTION

The present description is provided without limitation, each feature of one embodiment being able to be combined with any other feature of any other embodiment in an advantageous manner.

It should henceforth be noted that the figures are not to scale.

### Example of One Particular Implementation

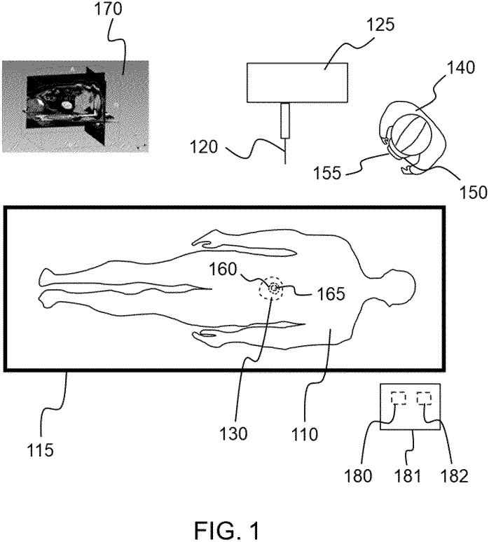

FIG. 1 is a schematic view of a medical intervention in which an individual 110 lying on a table 115 is treated using a medical instrument 120. In the present non-limiting example of the invention, the medical intervention corresponds to the ablation of a lesion 165 in an anatomical structure of interest 130, which in this case is the liver of the individual 110, by means of the medical instrument 120, which is in this case is a semi-rigid needle. The medical intervention in this case is a percutaneous procedure in which the body of the individual 110 is not opened up.

The manipulation of the medical instrument 120 by an operator 140 may advantageously be guided by means of a guiding device which in this non-limiting example of the invention is an augmented reality device such as a headset 150 worn by the operator 140. The medical instrument 120 may also be associated with a medical robotic device 125.

The headset 150 comprises a translucent screen 155 allowing the operator to see normally. On the screen 155, an image is overlaid in order to display markers that make it possible to guide the operator 140 in the manipulation of the medical instrument 120 with a view to ablation-treating a region 160, called the ablation region, around the lesion 165 identified in the anatomical structure of interest 130. The markers may in particular comprise an ablation mask which has been estimated beforehand on a medical image 170 of the anatomical structure of interest 130 acquired before the operation. The medical image 170 will hereinafter be called the pre-operative medical image 170.

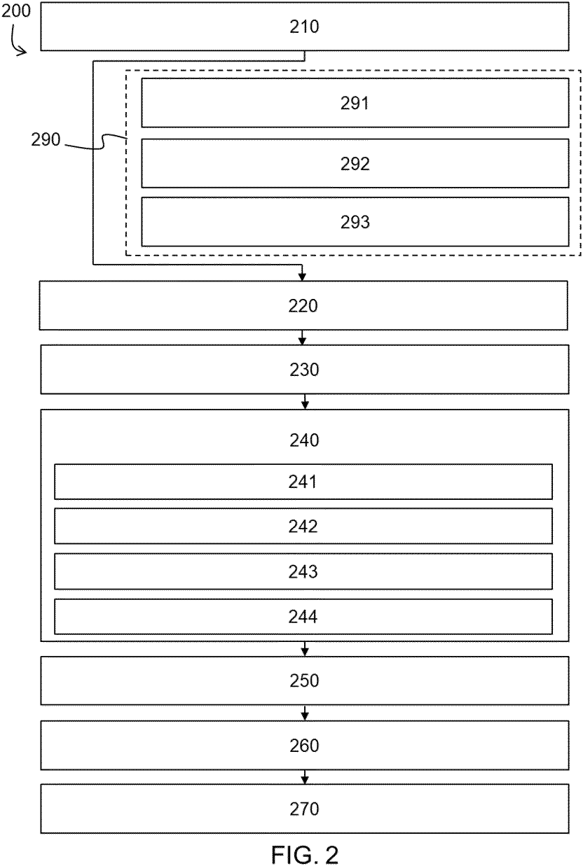

Once the operation is finished, an evaluation of the operative treatment is performed by a method 200 for the post-treatment evaluation of the ablation as illustrated in the form of a block diagram in FIG. 2 and the instructions for which are stored in a computer memory 180 of an electronic control device 181 connected to the headset 155 by cable or by wireless technology. The post-treatment evaluation method 200, the instructions for which are processed by a computer processor 182 of the electronic control device 181, makes it possible in particular to determine an ablation region and an associated risk of recurrence, in order to check whether the surgical treatment performed in the operation is sufficient or if it is preferable to continue the treatment by carrying out, for example, a supplementary ablation.

It should be noted that the electronic device 181 may advantageously be integrated into the headset 150.

The post-treatment evaluation method 200 comprises a first step 210 of acquiring a post-operative medical image of the anatomical structure of interest 130.

It should be noted that the pre-operative and post-operative medical images are preferably acquired by means of computed tomography. Alternatively, they may be acquired using a magnetic resonance imaging device.

Preferably, the technique used to acquire the pre-operative medical image and the post-operative medical image is similar, or even the same.

However, the technique used to acquire the post-operative medical image may be distinct from the technique used to acquire the pre-operative medical image.

The pre-operative and post-operative medical images are advantageously, in this non-limiting example of the invention, images acquired in three dimensions. In practice, each medical image acquired in three dimensions generally corresponds to a collection of medical images in two dimensions, each corresponding to a section through the anatomical structure of interest 130, taken at regular intervals along a predetermined axis. A three-dimensional representation of the anatomical structure of interest may be reconstructed from this collection of two-dimensional medical images. The term “three-dimensional image” will thus be understood to mean both a collection of medical images and a three-dimensional representation. The term “voxel” will be understood to mean an elementary unit relating to the resolution of the three-dimensional image.

Alternatively, the pre-operative and post-operative medical images are each acquired in two dimensions. The elementary unit relating to the resolution of the two-dimensional image is then commonly called a pixel.

The pre- and post-operative medical images are images comprising the entire anatomical structure of interest or are cropped around the ablation region using a predefined frame. In a three-dimensional image, the frame surrounding the ablation region corresponds to a cube, whereas in a two-dimensional image, the frame corresponds to a square.

The frame surrounding the ablation region, also known as the “bounding box”, may be generated automatically around the ablation region following an action by the operator. Such an action may, for example, correspond to the operator indicating a point in the post-operative medical image belonging to the ablation region and the frame is generated around this point. For example, in the context of a minimally invasive ablation treatment on small lesions, i.e. on lesions of the order of 5 cm +/-10% in maximum diameter, for example, or even preferably of the order of 3 cm +/-10% in maximum diameter, each edge of the cube or each side of the square measures between 5 and 10 cm.

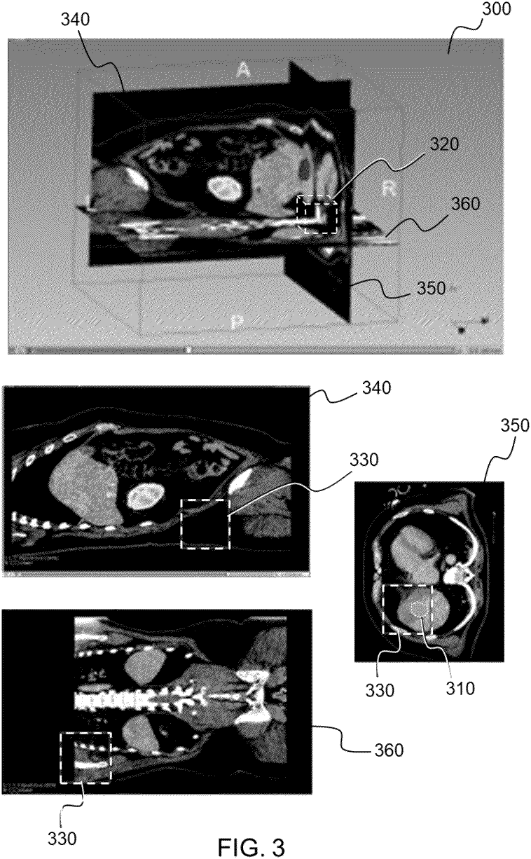

FIG. 3 is an illustration of a three-dimensional medical image 300 in which an ablation region 310 is surrounded by a frame 320. The frame 320 is a cube and corresponds to squares 330 in the sectional views 340, 350 and 360, which are a sagittal view, an axial view and a coronal view, respectively.

The post-operative medical image of the anatomical structure of interest 130 is then analyzed by a neural network, which is a machine learning method, in a second step 220 in order to automatically segment the ablation region in the post-operative medical image of the anatomical structure of interest 130 of the individual 110.

To that end, the neural network has previously been trained on a database of medical images of an identical anatomical structure of interest, in this case therefore a liver, of a set of patients in a preliminary training phase 290. Each medical image in the database comprises an anatomical structure of interest that has a function identical to that of the anatomical structure of interest 130.

Advantageously, the post-operative medical image of the anatomical structure of interest 130 of the individual 110 is acquired in the same way as for the medical images in the training database for the neural network.

When the post-operative medical image of the anatomical structure of interest 130 of the individual 110 is cropped, the dimensions of the cube or square of this post-operative medical image are advantageously identical to those of the cubes or squares used to train the neural network. In other words, the medical images in the database have the same dimensions as the cropped post-operative medical image.

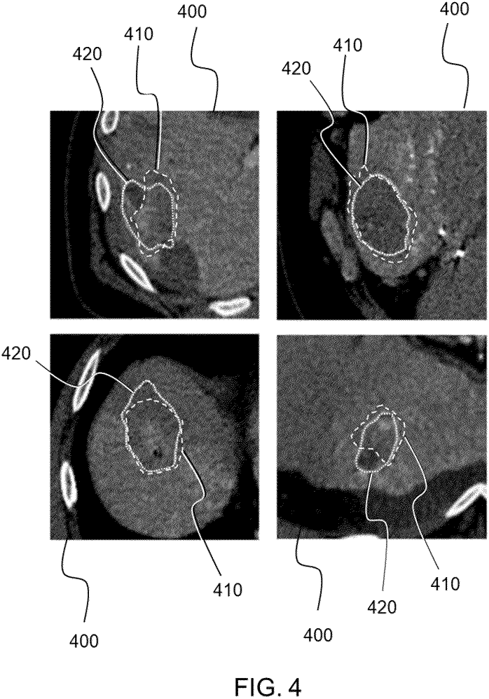

In order to train the neural network, the ablation region of each post-operative image in the database, where the lesion was ablated, has previously been segmented by at least two operators, in order to increase the relevance of the learning and therefore of the analysis results obtained by the neural network. Specifically, it may be difficult for an operator to delimit an ablation region, in particular when the contrast in the image is low, as may be seen, for example, in the four previously segmented post-operative medical images 400 in FIG. 4. The use of a plurality of operators to annotate the medical images therefore makes it possible to improve the identification of the ablation region. The ablation region associated with the registered post-operative image thus corresponds, in this non-limiting example of the invention, to the combination of the ablation regions proposed by the operators. As an alternative example, the ablation region associated with the registered post-operative medical image may correspond to the intersection, to a consensus or to an adjudication of the ablation regions proposed by the operators. The neural network is further trained to classify the voxels of a medical image in a region with ablation or without ablation.

Alternatively, the learning may be performed using a single expert annotator who delineates the ablation regions in the medical images. The operator’s experience is then important so that the neural network may arrive at well-defined ablation regions.

It should also be noted that it is preferable for the set of images in the database to comprise as many voxels belonging to the region with ablation as voxels belonging to a region without ablation. This proportion is calculated on the basis of the voxel classification determined manually by operators.

In other words, the portion of the individual’s body included in each image in the database is divided into a plurality of elementary units of a single size, the number of elementary units being divided into two near-equal parts between the portion of the human body delimited by the ablation region and the rest of the portion of the individual’s body included in the image.

Furthermore, it is also preferable for the ablation region not always to be in the center of the frame in every medical image in the training database. In the case of the ablation region being mainly in the center of the frame, a bias would be introduced into the neural network which would learn that the ablation region is mainly a region in the center of the frame, which is not necessarily the case, especially in the case of an error in the positioning of the frame by an operator. A bounded random variable is therefore advantageously added to the position of the frames in order to limit this bias regarding the positioning of the lesion in the center of the frame.

The set of the centers of the ablation region in the cropped medical images in the database thus form a constellation of distinct points inside the common frame.

In order to limit the generation of false positives by the neural network, it is further preferable for the database to comprise medical images comprising at least one unablated lesion.

Phase 290 of training the neural network is generally carried out in several steps:
a training step 291;
a validation step 292;
a test step 293.

The medical image database is thus partitioned into three databases comprising distinct medical images. The three databases are called the training database, the validation database, and the test database, respectively. In this non-limiting example of the invention, 60 to 98% of the medical images in the medical image database are grouped together in the training database, 1 to 20% in the validation database and 1 to 20% in the test database. The percentages, which generally depend on the number of images in the medical image database, are given here by way of indication.

The first two steps, 291 and 292, of phase 290 of training the neural network are main steps which may be repeated a number of times. The third test step is optional.

In the first step 291 of training phase 290, a weight W and a bias b for each neuron of the neural network are determined on the basis of the medical images in the training database.

It should be noted that the training database may advantageously comprise medical images comprising at least one unablated lesion.

The second step 292 in training phase 290 makes it possible to validate the weight W and the bias b determined beforehand for each neuron of the neural network, on the basis of the medical images in the validation database, in order to verify the results of the neural network, in particular the prediction error, i.e. by comparing, per medical image in the validation database, the ablation region obtained with the ablation region segmented in the medical image extracted from the training database.

In the case of the prediction error being too great on completion of this second step, the two training 291 and validation 292 steps are implemented again to re-train the neural network re-using the same medical images, in order to refine the values of the weights W and of the biases b for each neuron.

Alternatively, when re-training the neural network, the first step 291 uses re-sampling of the medical images, taking into consideration for the training the medical images in the training database and some of the medical images in the validation database. The rest of the medical images in the validation database are then used to validate the weights W and the biases b obtained on completion of the first re-training step.

It should be noted that the neural network may be re-trained as many times as necessary until the prediction error is acceptable, i.e. is lower than a predetermined value.

Once the two steps 291 and 292 in training phase 290 have been implemented at least once, the final performance of the neural network may be tested in a possible third test step 293 using the medical images in the test database. These medical images, distinct from the medical images in the training and validation databases, make it possible to verify that the neural network as configured with the parameters W and b for each neuron makes it possible to segment the ablation region with a high degree of precision in all situations with which the neural network is likely to be confronted. However, unlike validation step 292, this potential third test step 293 does not result in a new training cycle for the neural network.

It should be noted that the images used in test step 293 are generally carefully selected in order to cover different positions and sizes of the ablation region in the anatomical structure of interest in order to best test the predictive abilities of the neural network.

On the basis of the post-operative medical image of the anatomical structure of interest 130, the neural network classifies each voxel as a region with ablation or without ablation. This prediction may take the form of an ablation mask overlaid over the post-operative medical image of the anatomical structure of interest 130. The ablation mask is generally registered on the voxels belonging to the ablation region predicted by the neural network. It should be noted that the ablation mask is usually delimited by a surface or by an outline in the context of a two-dimensional image.

Advantageously, before training phase 290, the neural network may have been trained beforehand, in what is called a pre-training phase 295, on a second database of medical images comprising medical images showing a lesion of an anatomical structure of interest of the same type as that of the individual 110. This pre-training phase 295 allows for better segmentation of the ablation region in the post-operative medical image of the individual 110, ingeniously using the similarity in shape of a lesion and of an ablation region, or even in position within the anatomical structure of interest.

It should be noted that the medical images in the second database may advantageously have been segmented beforehand by at least one operator. The learning in phase 295 is similar to that performed in phase 290.

The post-operative medical image is registered with the pre-operative medical image 170 in a third step 230 of the method 200 illustrated in FIG. 2. The registration, which makes it possible to find correspondences between anatomical points in the two medical images, is carried out using a method known to those skilled in the art. The registration may be carried out rigidly, i.e. all of the points in the images are transformed in the same way, or non-rigidly, i.e. each point in the images may have a specific transformation.

An evaluation of a risk of recurrence is then carried out in a fourth step 240 comprising four sub-steps 241, 242, 243 and 244.

In a possible sub-step 241, the lesion 165 is detected in the pre-operative medical image of the anatomical structure of interest 130 of the individual 110. This detection may be performed automatically, or manually by an operator.

In sub-step 242, segmentation of the lesion 165 is performed automatically on the pre-operative medical image of the anatomical structure of interest 130 of the individual 110. Alternatively, the segmentation is performed manually by an operator.

The lesion is segmented automatically using methods known to those skilled in the art. For example, the segmentation is performed using a method based on the histogram of the image, such as, for example, Otsu’s method, or using a deep learning method.

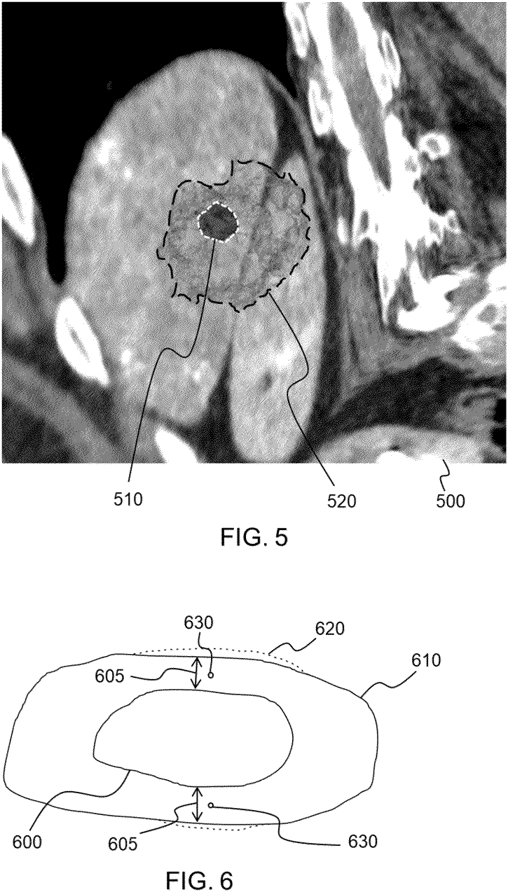

This segmentation sub-step 242 is illustrated by FIG. 5 which shows a pre-operative medical image 500 in which automatic segmentation based on a deep learning method is performed in order to determine the three-dimensional location of the lesion 510 and of the ablation region 520. An equivalent result may be obtained using a neural network distinct from the neural network used in step 230.

An ablation margin is then determined between the segmentation of the lesion and the ablation mask established previously, in sub-step 243. The ablation margin corresponds to the minimum margin, i.e. the minimum distance, taken between the segmentation of the lesion and the ablation mask. In other words, the ablation margin corresponds to the smallest distance calculated between a point of the lesion and a point of the ablation region and is calculated for all of the points of the lesion.

Determining the ablation margins makes it possible to ensure that the ablation region properly covers the lesion.

On the basis of the value of the ablation margin determined in sub-step 243, a prediction of a risk of recurrence or a determination of the position of the recurrence may be evaluated in sub-step 244 by comparing the calculated ablation margin with reference values of the ablation margins associated with a recurrence status, stored in a database, the recurrence status indicating whether or not a recurrence was observed after the operation, potentially with an associated date of recurrence. For example, it may be considered that the risk of recurrence is zero when the ablation margins are equal to or larger than 5 mm.

It should be noted that the prediction of a risk of recurrence of the lesion generally takes the form of a binary value equal, for example, to 0 (zero or negative risk) or to 1 (demonstrated or positive risk).

In addition or alternatively, predictors of the risk of recurrence other than ablation margins may be used. The risk of recurrence and the position of the recurrence may be estimated by weighting all or some of these different predictors. For example, the predictors of the risk of recurrence are based on relative characteristics which may be:
an ablation margin;
a distance between the surface of the lesion, or part of the surface of the lesion, and the ablation region;
a distance between the centers of mass of the lesion and the centers of mass of the ablation region;
a distance between the surface of the lesion, or part of the surface of the lesion and the ablation region and the distance between the centers of mass of the lesion and the centers of mass of the ablation region taking account of the proximity of the capsule to the anatomical structure of interest, in particular in the case of sub-capsular lesions;
evenness of the edges of the ablation region in relation to the surrounding healthy tissue;
sharpness of the edges of the ablation region in relation to the surrounding healthy tissue;
a ratio of the volume of the lesion to the volume of the ablation region;
a position of the lesion in the anatomical structure of interest.

The reference value of the center of mass depends on the ablation margins. If the ablation margins are 10 mm and the reference value of the ablation margins is 5 mm, the distance between the centers of mass of the lesion and the centers of mass of the ablation region should be smaller than or equal to 5 mm.

It should be noted that the value of the risk of recurrence may advantageously be continuous between 0 and 1 instead of being a binary value, in order to take a plurality of risk predictors into consideration. A weighting may thus be performed between different risk predictors in order to obtain a value between 0 and 1. The risk of recurrence will then be understood as having been demonstrated when it is higher than a threshold value, for example equal to 0.5.

If the risk is demonstrated, the position of the recurrence may be determined in order to estimate a supplementary ablation mask in a fifth step 250 of the post-treatment evaluation method 200. The supplementary ablation mask is, for example, considered to perform a supplementary ablation in a region where the ablation margin is smaller than a given value, for example five millimeters.

In step 250, automatic identification of a region where the ablation margin is smaller than a threshold value, for example smaller than five millimeters, may be performed.

FIG. 6 illustrates an example of a supplementary ablation following ablation treatment of a lesion 600 with risk of recurrence. The evaluation method 200 has identified regions with insufficient ablation margins 605 between the lesion 600 and the ablation region 610, and has generated a supplementary ablation mask 620. It should be noted that the supplementary ablation mask 620 is generated while attempting to limit the ablation region as much as possible. In addition, target points 630 to be reached by an ablation needle can then be defined in mask 620.

The method 200 may also comprise a step 260 of planning a path to be followed by the medical instrument 120 associated either with the ablation mask or with the supplementary ablation mask, in order to guide the operator in the manipulation of the medical instrument 120 in a step 270 of guiding the medical instrument 120 along the planned path.

One example of a planning method is described in French patent application no. 1914780 entitled “Method for automatically planning a trajectory for a medical intervention”.

It should be noted that the guidance in this non-limiting example of the invention is visual, by displaying the planned path and/or a guiding indicator on the screen 155 of the headset 150.

Alternatively, the medical instrument 120 may be guided by means of a navigation system providing information on the position and orientation of the medical instrument 120. It may be a case of mechanical guidance via a robotic device coupled to such a navigation system.

It should be noted that steps 230 to 260 may be repeated until the risk of recurrence is zero or almost zero, or until the ablation margins are sufficient.

1. A method for the post-treatment evaluation of an ablation of a portion of an anatomical structure of interest of an individual, the anatomical structure of interest comprising at least one lesion, the ablation of the portion of the anatomical structure of interest being delimited by an ablation region, the evaluation method comprising the steps of:
acquiring a post-operative medical image of the anatomical structure of interest of the individual, comprising all or part of the ablation region; and
automatically determining an outline of the ablation region via a machine learning method, of neural network type, analyzing the post-treatment image of the anatomical structure of interest of the individual, said machine learning method being trained beforehand in a training phase using a database comprising a plurality of post-operative medical images of an identical anatomical structure of interest of a set of patients, each medical image in the database being associated with an ablation region for the anatomical structure of interest of said patient.

2. The post-treatment evaluation method of claim 1, wherein the training phase comprises a prior step of training using medical images of an identical anatomical structure of interest comprising an unablated lesion.

3. The post-treatment evaluation method of claim 1, further comprising a step of registering the post-operative medical image and a pre-operative medical image of the anatomical structure of interest of the individual.

4. The post-treatment evaluation method of claim 1, further comprising a step of evaluating a risk of recurrence according to a relative characteristic between the ablation region and the lesion, between the ablation region and the anatomical structure of interest or between the lesion and the anatomical structure of interest.

5. The post-treatment evaluation method of claim 4, further comprising, when the risk of recurrence is demonstrated, a step of determining the position of the recurrence according to a relative characteristic between the ablation region and the lesion, between the ablation region and the anatomical structure of interest or between the lesion and the anatomical structure of interest.

6. The post-treatment evaluation method of claim 4. wherein a relative characteristic is an ablation margin between the ablation region and the lesion.

7. The post-treatment evaluation method of claim 1, wherein a relative characteristic is a center of mass of the lesion and a center of mass of the ablation region.

8. The post-treatment evaluation of claim 4. wherein a relative characteristic is the evenness and sharpness of the edges of the ablation region in relation to the surrounding healthy tissue.

9. The post-treatment evaluation method of claim 4, wherein a relative characteristic is the ratio of the volume of the lesion to the volume of the ablation region.

10. The post-treatment evaluation method of claim 4. wherein a relative characteristic is a position of the lesion in relation to the center of the anatomical structure of interest.

11. The post-treatment evaluation method of claim 3, further comprising a step of segmenting the lesion in the pre-operative medical image of the anatomical structure of interest of the individual.

12. The post-treatment evaluation method of claim 3, further comprising a step of detecting the lesion in the pre-operative medical image of the anatomical structure of interest of the individual.

13. The post-treatment evaluation method of claim 3, as wherein all or some of the training post-operative medical images in the database are cropped around the ablation region comprising at least one lesion, the cropping of the images being carried out using a common frame of predetermined size, the set of the centers of the ablation region in the cropped post-operative medical images forming a constellation of distinct points inside the common frame.

14. The post-treatment evaluation method of claim 3, wherein for the set of post-operative medical images in the database, the portion of the individual’s body included in the image is divided into a plurality of elementary units of a single size, the number of elementary units being divided into two near-equal parts between the portion of the human body delimited by the ablation region and the rest of the portion of the individual’s body included in the image.

15. The post-treatment evaluation method of claim 3, wherein the post-operative medical image database comprises at least one pre-operative medical image comprising at least one unablated lesion.

16. The post-treatment evaluation method of claim 4. further comprising a step of determining a supplementary ablation mask when the risk of recurrence is demonstrated.

17. The post-treatment evaluation method of claim 16, further comprising a step of proposing a path to be followed by a medical instrument to a target point of the supplementary ablation mask.

18. The post-treatment evaluation method of claim 1, wherein the medical images are three-dimensional images.

19. The post-treatment evaluation method of claim 3. wherein each post-operative image is acquired using the same image acquisition technique.

20. An electronic device comprising a processor and computer memory storing instructions for a post-treatment evaluation method of claim 1.

21. The electronic device of claim 20, wherein said electronic device is a control device, a navigation system, a robotic device or an augmented reality device.
